(19) United States
(12) Patent Application Publication
(10) Pub. No.: US 2018/0296450 A1
(43) Pub. Date: Oct. 18, 2018

(54) DENTAL WHITENING COMPOSITIONS

(71) Applicants: Vivekananda Ramana, East Stroudsburg, PA (US); Joel Sterling Douglas, Bonita Springs, FL (US)

(72) Inventors: Vivekananda Ramana, East Stroudsburg, PA (US); Joel Sterling Douglas, Bonita Springs, FL (US)

(21) Appl. No.: 15/485,247

(22) Filed: Apr. 12, 2017

(51) Int. Cl. A61K 8/25; A61K 8/24; A61K 8/19; A61K 8/21; A61Q 11/00; A61K 8/02; A61K 8/34

(52) U.S. Cl. CPC A61K 8/25; A61K 8/24; A61K 8/19; A61K 8/21; A61K 2800/412; A61K 8/0245; A61K 8/34; A61K 2800/28; A61K 2800/884; A61Q 11/00

(57) ABSTRACT

Provided are methods and compositions relating to a dental whitening compositions

### DESCRIPTION CROSS REFERENCE TO RELATED

Applications None

### STATEMENT REGARDING FEDERALLY SPONSORED RESEARCH OR DEVELOPMENT

The research for the Patent “DENTAL WHITENING COMPOSITIONS” was not funded by any federally sponsored research or development.

### BACKGROUND OF THE INVENTION

White teeth have long been considered cosmetically desirable. Unfortunately, teeth almost invariably discolor. Over time, use of certain foods and tobacco, the process of aging, diseases, trauma, medications, some congenital conditions, and environmental effects cause teeth to discolor to varying degrees. This inevitable discoloration, combined with the desire for whiter teeth, have led to a high level of interest in developing compositions and methods for bleaching teeth. To this end, people have historically relied on mechanical cleaning methods, having veneers placed over their teeth, or having their teeth chemically bleached to achieve a desired level of tooth whiteness. These attempts at the restoration of tooth enamel for cosmetic effect is widespread in the prior art. The systems and methods used, if done improperly, can damage the tooth enamel, as well as damage the underlying gum structure.

Additionally, tooth enamel is the first line of defense against hot, cold, sticky, and abrasive items. When tooth enamel wears down, it can cause tooth decay, exposing dentin and sensitive nerves in the pulp of the teeth, and causing the tooth to appear yellow in color. The enamel can weaker with age, a diet high in sugar content or acidity, and/or a history of acid reflux disease.

The vast majority of tooth bleaching systems currently in use employ a form of peroxide as a whitening agent. The efficacy of peroxide compounds for improving oral hygiene has long been recognized. Dental clinicians have used peroxide for several decades as an oral antiseptic. Such compounds have proven effective in the treatment of gingivitis, oral lesions, periodontitis, and herpetic stomatitis, and in combating plaque. The majority of tooth whitening compounds in use currently employ carbamide peroxide (CO(NH2)2H2O2), also called urea hydrogen peroxide, hydrogen peroxide carbamide, and perhydrol-urea, as the whitening agent. Also, peroxide salts of the alkali or alkaline earth metals are known to be useful in bleaching teeth.

U.S. Pat. Nos. 5,098,303, 5,234,342, 5,376,006, and 5,725,843, herein incorporated by reference, teach water-based bleaching gels that contain carbomer, glycerin, and a peroxide such as hydrogen peroxide or carbamide peroxide (urea peroxide). In addition, U.S. Pat. No. 3,657,413, herein incorporated by reference describes a bleaching composition that contains urea peroxide.

Further examples of tooth bleaching compositions containing peroxides can be found in the disclosures of U.S. Pat. Nos. 4,839,157 and 4,405,599, herein incorporated by reference. These compositions included various abrasive agents such as dicalcium phosphate, calcium carbonate, magnesium carbonate, silica, or polyethylene compounds. The use of abrasive constituents in dentifrices containing peroxide compounds results irritation to both tooth and gum surfaces that is further compounded by the interaction of the peroxide composition on the abraded surfaces.

U.S. Pat. Nos. 5,098,303, 5,376,006 and 5,725,843, herein incorporated by reference teach high viscosity sustained release dental compositions, such as tooth bleaching or fluoride compositions, for treating tooth surfaces. The sustained release dental compositions include a high carboxypolymethylene concentration (typically greater than 3.5%) resulting in a very high viscosity. Bleaching gels can contain from about 3% to about 20% carbamide peroxide, preferably about 4% to about 15% carbamide peroxide. Alternatively, bleaching gels can contain hydrogen peroxide in a preferred range from about 2% to about 10%.

U.S. Pat. No. 4,226,851 herein incorporated by reference teaches a stable dental hygiene compositions comprising a mixture of hydrogen peroxide and zinc chloride. The mixture is stabilized by the addition of watet soluble vitamin E. Opalescence® PF produced by Ultradent Products, Inc. of South Jordan, Utah, offers four concentrations (10%, 15%, 20%, and 35%) of carbamide peroxide with potassium nitrate and fluoride for treatment flexibility. Available in three flavors, the gel contains 20% water to prevent dehydration, and the sticky, viscous formula helps keep the gel and tray in place.

None of the aforementioned references describes the use of bioactive glass applied to the existing exposed dentin due to loss of enamel that causes the tooth to appear yellow. The use of bioactive glass will be shown to retard the effect of staining and provide a mechanism to enhance light reflection, so the tooth appears whiter than the native dentin exposed due to enamel loss.

However, glasses having a bioactive and, also, sometimes antimicrobial effect are described as bioglass in An Introduction to Bioceramics, World Scientific Pub I: (Hensch and Wilson, 1993). Bioglass of this type is distinguished by the formation of hydroxylapatite layers in aqueous media. Heavy metal-free alkali and alkaline earth silicate glasses having antimicrobial properties are described in the applications DE-A-199 32 238 and DE-A-199 32 239, herein incorporated by reference.

A glass powder that includes 40-60 weight-percent SiO.sub.2, 5-30 weight-percent Na.sub.2O, 10-35 weight-percent CaO, and 0-12 weight-percent P2Os is known from U.S. Pat. No 5,676,720 herein incorporated by reference, a glass ceramic manufactured from a glass of this type of composition also being known. However, no information about the crystal phase is given in U.S. Pat. No. 5,676,720.

U.S. Pat. No. 5,981,412, herein incorporated by reference describes a bioactivebioceramic for medical applications having the crystalline phase Na2O.2CaO.3SiO2. The crystallite size is 13 μm. The ceramization is performed using tempering steps for nucleation and crystallization. The emphasis is on mechanical properties such as K1c. The crystal phase component is between 34 and 60 volume-percent. U.S. Pat. No. 5,981,412, herein incorporated by reference only describes a crystalline phase that is a high-temperature phase and that only arises under the special conditions specified in this publication. An application in the field of dental care is not described. U.S. Pat. Application 200901977221, herein incorporated by reference describes dental bonding compositions and U.S. Pat. Application 20050142077, herein incorporated by reference describes the use of antimicrobial glass ceramic in dental care.

The use of bioactive glasses for toothpaste and gels is described, for example, in WO 97/27148, herein incorporated by reference. Inorganic non-metallic materials that contain calcium and phosphorus and lead to tooth remineralization through appropriate ion donations are known from U.S. Pat. Nos. 5,427,768 and 5,268,167, herein incorporated by reference.

The use of the glasses cited above in the fields of dental care and/or oral hygiene do not identify the ability for the glass to be used to increase the perception of whiteness of the tooth. Additionally, all the above publications and patents cited in this specification are herein incorporated by reference as if each individual publication or patent were specifically and individually indicated to be incorporated by reference and are incorporated herein by reference to disclose and describe the methods and/or materials in connection with which the publications are cited.

The instant invention solves the problems of using peroxides by creating a whitening composition formed from bioactive glass and a suitable non-aqueous solvent, for the first time, an effective whitening method is provided that does not utilize excessive bleaching or contains a large concentration of hydrogen peroxide.

### SUMMARY OF THE INVENTION

The disclosure provides methods and compositions relating to a whitening composition useful in whitening teeth. Teeth are protected by enamel, their first defense against hot, cold, sticky, and abrasive items. When enamel wears down, it can cause tooth decay, exposing dentin and sensitive nerves within the pulp of the teeth, and causes the tooth to look yellow. Enamel can become weaker with age, an acidic or sugary diet, and/or a history of acid reflux disease. The invention focuses on the dentinal tubule, one of the very small tubes or canals in the dentin. These extend from the pulp cavity of the tooth to the enamel and are occupied by odontoblastic processes and occasional nerve filaments. One way to protect the dentin and tooth pulp cavity, and provide a brighter white appearance, is to fill the dentinal tubule with a reflective medium that will provide a white appearance of the tooth. In addition to occluding the dentin tubule with a micro mechanical bond, it is long known that the bioglass can, in the aqueous presence of calcium and phosphate, form apatite to further prevent sensitivity and lock out micro-leakage through the tubules and increase the appearance of whiter enamel structure.

Accordingly, the present disclosure provides methods for whitening teeth that have been affected by the loss of enamel. In certain embodiments, such methods include, first applying a diluted hydrogen peroxide mixture to the teeth, and then, applying an etching composition comprising an etchant to a tooth to etched the exposed dentin surface that has been exposed by the loss of enamel; applying a non-aqueous solvent composition to the etched dentin surface: the non-aqueous solvent composition includes a bioactive glass substantially lacking silanol groups and a non-aqueous solvent to the primed exposed dentin surface; and where the non-aqueous solvent composition provide for formation of a hybrid layer, where the hybrid layer comprises dentin and the bioglass composition.

The acid etch step in the tooth restoration process exposes the dentin tubules. The diameter of the open tubules measures about 1 μm. To allow penetration into the tubules, the bioglass has been manufactured to a particle size of less than 1 μm maximum. At this size, the particles are found to penetrate deeper into the tubules creating a reflective surface that will appear white when exposed to normal lighting conditions. In addition to occluding the dentin tubule with a micro-mechanical bond, it is long known that the bioglass can, in the aqueous presence of calcium and phosphate, form apatite to further provide a perception of tooth whitening.

Also provided are methods for whitening teeth that have damaged enamel. In certain embodiments, such methods include applying an etching composition comprising an etchant to a tooth to produce an etched dentin surface; applying a bioactive glass composition comprising a bioactive glass substantially lacking silanol groups and a non-aqueous solvent. Such methods can provide for formation of a hybrid layer, where the hybrid layer comprises dentin and the dental whitening composition. In certain embodiments, such methods include that the bioactive glass is present at about 0.5% to about 40% by weight percentage of the bioactive glass composition.

Also provided are dental whitening compositions that include a bioactive glass substantially lacking silanol groups and a non-aqueous solvent comprising an alcohol. Ideally, a resin-based adhesive can be used after the application of the dental whitening composition as part of the restoration of teeth. In exemplary embodiments, the bioactive glass has the following approximate composition by weight percentage: SiO2 (49.5%), Na2O (17%), Cao (26.9%), P2Os (6.6%). In further embodiments, the bioactive glass has an average particle size of 1 μm or less. In still further embodiments, the dental whitening composition has about 0.5 to about 1% by weight of said bioactive glass. In an exemplary embodiment, the alcohol solvent is ethanol. These whitening compositions are also useful in dental restoration when applied under a dental resin composite used to restore the tooth they can make the restored area appear whiter.

Also provided are kits containing the dental whitening composition for use in the subject methods. In certain embodiments the kits may include a whitening composition that comprises a bioactive glass substantially lacking silanol groups and a non-aqueous solvent comprising an alcohol, and, optionally, an etching composition. In further embodiments, the kit may include the dental whitening composition provided as separate components, where a first component comprises the bioactive glass in the non-aqueous solvent, where the first component is provided in a container. In further embodiments the kit may include the dental whitening composition provided as separate components, where a first component comprises the bioactive glass and the second container the non-aqueous solvent.

### BRIEF DESCRIPTION OF THE FIGURES

FIG. 1 shows scanning electron microscopy (SEM) micrographs untreated dentin.

FIG. 2 shows SEM micrographs dentin treated with bioglass.

### DEFINITIONS

The term “dentin” used herein refers to a calcified tissue of the body that is one of the major components of teeth. Dentin is usually covered by enamel that forms the outer surface of the tooth. Dentin is a porous matrix composed of up to 70% hydroxyapatite. Dentin has microscopic channels called dentinal tubules that span the thickness of the dentin. Dentinal tubules taper in diameter from the inner to the outermost surface of the dentin having a diameter of about 2.5 μm near the inner surface of the dentin, about 1.2 μm in the middle of the dentin and about 900 nm near the outer surface of the dentin. In addition, dentinal tubules are surrounded by collagen fibers that form an extensive collagen network.

The term “etch” or “etching” as used herein means applying an acid to the surface of a tooth to partially dissolve the apatite and produce irregularities in the surface of dentin.

The terms bioglass and “bioactive glass” are used interchangeably.

The term “hybrid layer” where the hybrid layer comprises dentin and bioglass composition.

The terms “substantially lacks” or “substantially lacking” as used herein refer to a compound that is at least about 60% free, or about 75% free, or about 90-95% free from a component. For example, “substantially lacking silanol groups” refers to a compound that is at least about 60% free, or about 75% free, or about 90-95% free of silanol groups.

The term “non-aqueous solvent” is meant to encompass solvents that do not contain water as a predominant component and includes solvents that contain, for example, less than 15% water by volume, less than 10% water by volume, less than 5% water by volume, less than 1% water by volume, and may contain no detectable water.

### DETAILED DESCRIPTION OF THE INVENTION

Exemplary embodiments of the methods and compositions of the present disclosure are provided below. These include methods of utilizing bioglass to increase the whiteness of teeth affected by the erosion of their enamel, and kits useful in such methods.

Before the present invention is described in greater detail, it is to be understood that this invention is not limited to embodiments described, as such may, of course, vary. It is also to be understood that the terminology used herein is for the purpose of describing embodiments only, and is not intended to be limiting, since the scope of the present invention will be limited only by the appended claims.

Where a range of values is provided, it is understood that each intervening value, to the tenth of the unit of the lower limit unless the context dearly dictates otherwise, between the upper and lower limit of that range and any other stated or intervening value in that stated range is encompassed within the invention.

Unless defined otherwise, all technical and scientific terms used herein have the same meaning as commonly understood by one of ordinary kill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

It must be noted that as used herein and in the appended claims, the singular form “a”, “an”, and “the” include plural referents unless the context clearly dictates otherwise. It is further noted that the claims may be drafted to exclude any element including optional elements. As such, this statement is intended to serve as antecedent basis for use of such exclusive terminology as “solely”, “only”, and the like in connection with the recitation of claim elements, or use of a “negative” limitation.

As will be apparent to those of skill in the art upon reading this disclosure, each of the individual embodiments described and illustrated herein has discrete components and features that may be readily separated from or combined with the features of any of the other several embodiments without departing from the scope or spirit of the present invention. Any recited method can be carried out in the order of events recited or in any other order that is logically possible.

Dental whitening compositions of the present disclosure will be described first, followed by a detailed description of exemplary uses for the dental whitening compositions.

### Dental Whitening Compositions

A dental whitening composition for application to dentin is provided. The term “dental whitening compositions” is intended to refer to any bioactive glass-containing composition that can find a use in one or more steps of a whitening method according to the present disclosure. As noted earlier, tooth discoloration occurs when enamel wears down, it can cause tooth decay, exposing dentin and sensitive nerves within the pulp of the teeth, and causes the tooth to appear yellow in color.

Thus, dental whitening compositions include, but are not necessarily limited to, compositions comprising a suitable non-aqueous solvent (e.g., an alcohol such as ethanol) and bioactive glass (e.g., to be applied to an etched dentin surface). The bioactive glass present in the dental whitening composition is generally a bioactive glass that substantially lacks silanol groups. Exemplary dental whitening compositions are described in more detail below.

In certain cases, the dental whitening composition can include a bioactive glass and a suitable non-aqueous solvent. In some cases, the dental whitening composition can comprise from about 0.5% to about 10% by weight, about 0.5% to about 5% by weight, or about. about 1% by weight of the bioactive glass. In cases where a higher weight % of the bioactive glass is described, suspension of the bioactive glass in suitable non-aqueous solvent may be facilitated by use of a bioactive glass powder having a smaller average particle size (e.g., less than 1 μm average particle size, preferably 0.5 μm).

In one embodiment, the dental whitening composition is provided as bioactive glass suspended in a suitable non-aqueous solvent (e.g., a slurry). In these embodiments, the dental whitening composition comprises about 5% by weight, about 10% by weight, about 15% by weight, about 20% by weight, about 25% by weight. about 30% by weight, about 35% by weight, or about 40% by weight, or more, of the bioactive glass. The amount of bioactive glass incorporated into the dental whitening composition can vary with the average particle size of the bioactive glass. Smaller average particle sizes (e.g., 1 μm or less) may allow for more bioactive glass to be suspended in the dental whitening composition mixture.

In certain case, the bioactive glass used in the dental whitening composition is Bioglass 45S5 and has the following approximate composition by weight percentage: SiO2 (49.5%), Na2O (17%), CaO (26.9%), P2Os (6.6%). In other case the bioactive glass used in the dental whitening composition is Biogiass F glass and has the following approximate composition by weight percentage: SiO2 (44%), Na2O (23%), Cao (10%), MgO (4.5%), P2Os (6%) CaF2 (12.5%). In either case, the bioactive glass used in the dental whitening composition may have a average particle size of 1 μm or less and preferably 0.5 μm. Bioglass 45S5 or Bioglass F powders may be prepared by methods known to those of skill in the art, including but not limited to planetary ball milling of Bioglass 45S5 glass chips.

The bioactive glass is dispersed in the dental whitening composition via a solvent. As discussed below, the water content of the solvent is selected so that reaction of the bioactive glass with water in the solvent is insignificant, and may be so low as to avoid such reaction.

The key to the dental whitening composition performance is the small particle size distribution, 1 μm maximum of the bioglass component. The acid etch step in the tooth restoration process exposes the dentinal tubules. The diameter of the open tubules measures about 1 μm, therefore, the bioglass must have a maximum dimension of less than 1 μm, preferably 0.5 μm. To allow penetration into the dentinal tubules, bioglass needs to be manufactured to a particle sire of less than 1 μm and preferably 0.5 μm. At this size, the particles are found to penetrate deeper into the tubules and will cause the reflection of light to appear more white in color. In addition to occluding the dentinal tubule with a micro-mechanical bond, it is long known that the bioglass can, in the aqueous presence of calcium and phosphate, form apatite to further prevent sensitivity, lock out micro-leakage through the tubules, and increase the appearance of a whiter color enamel structure.

The dental whitening composition theoretically has the added benefit of facilitating the inhibition of leakage of particulate materials and/or fluid from the dentin or oral environment treated with the dental whitening composition. Inhibition of leakage can include both microleakage and nanoleakage. Microleakage is the seepage of fluids, debris, and/or microorganisms (e.g., bacteria) into micrometer-sized gaps (approximately 10-⁶ m) between any dental restoration and a tooth. Nanoleakage is the seepage of fluids, debris, and/or microorganisms (e.g. bacteria) into nanometer-sized gaps (i.e., approximately 1-0⁹ m) between any dental restoration and a tooth. Without being held to theory, the ability of bioactive glasses to promote the formation of apatite in aqueous environments that contain calcium and phosphate (e.g., saliva) can facilitate inhibition of leakage at the bonded interface through a mechanism of self-sealing due to the formation of apatite. This sealing prevents the intrusion of materials that would further discolor the tooth.

### Bioactive Glass Compounds

Bioactive glasses elicit a series of chemical reactions when they are brought into contact with an aqueous environment that contains calcium and phosphate, such as bone or tissue, leading to the formation of carbonated hydroxyapatite (HCA), similar to the mineral that forms teeth. The formation of HCA creates a bond between the bioactive glass and the dentin. The bond may be a mechanical bond and/or due to a chemical interaction between the bioactive glass and the dentin, forming HCA bonds to specific amino acids the collagen matrix of the dentin.

Bioactive glasses may contain but are not limited to, silicon dioxide (SiO2), sodium oxide (Na2O), calcium oxide (CaO), magnesium oxide (MgO), phosphorous pentoxide (P2Os) and calcium fluoride (CaF2). Other components may be added, such as boron, magnesium, aluminum iron, titanium, fluorine, and silver. The addition of fluorine to bioactive glass can be rationalized for the following reasons: firstly, that the rate of apatite formation at the glass surface can be enhanced; and secondly, that the apatite formed will be less vulnerable to acid attack; both are desirable in bioactive glass to be utilized in dental applications. Magnesium may also be added to bioactive glass, as it has been shown to slow down the rate of apatite precipitation, thus leading to more controlled mineralization.

Bioactive glass for use in the positions and methods disclosed herein generally are characterized by having predominantly more silicon dioxide (SiO2) groups than silanol (H3SiOH) groups. This improves the whitening appearance, because the SiO2 color for SiO2 in the 150 A range is light blue, perceived as whiter than the yellow color dentin. In general, this can be achieved by avoiding bioactive glass contact with water and/or hydrogen atoms, thereby inhibiting production of silanol in the bioactive glass composition (e.g., as by the reaction Si-0-Na++H++OH—; Si—OH++Na++OH—). Accordingly, bioactive glass can be described as having as a total of Si02 and H3SiOH groups, greater than 50% Si02 groups, greater than 60% Si02 groups, greater than 75% Si02 groups, greater than 85% Si02 groups, greater than 90% Si02 groups, greater than 95% Si02 groups, and can be described as having, as a total of Si02 and H3SiOH groups, at least 55% Si02 groups, at least 65% Si02 groups, at least 80% Si02 groups, at least 90% Si02 groups or more. In some embodiments, the bioactive glass is characterized as “substantially lacking silanol groups,” as outlined above, refers, to a bioactive glass that is at least about 60% free, or about 75% free, or about 90-95% free of silanol groups.

Bioactive glass compositions can be maintained in a relative “dehydrated” state prior to use. The term “dry” as used herein can refer to compositions kept at ambient conditions, for example, at standard temperature, pressure, and humidity. It should be noted, however, that a “dry” compound can be provided in a substantially non-aqueous solvent (e.g., an alcohol). Thus, for example slurries of bioactive glass powder in suitable non-aqueous solvent (e.g., an alcohol (e.g. ethanol)) are encompassed within the meaning of a “dry” bioactive glass composition. In some cases, the bioactive glass compositions may be stored in suitable packaging to keep the bioactive glass compositions dry (e.g. a sealed container).

Bioactive glass for use in the compositions and methods disclosed herein can be selected to have an average particle size that allows the bioactive glass particles to penetrate into the lumens of dentinal tubules. Oentinal tubules generally have diameters of approximately 0.8 μm to 1 μm before etching, and may have diameters of approximately 1 μm or greater after etching. Accordingly, suitable bioactive glass compositions for incorporation in the whitening compositions of the present disclosure include those having an average particle size of 1 μm or less, preferably 0.5 μm.

Bioactive glass compositions can have a particle size distribution of at least 25%, at least 50%, at least 75%, at least 85%, or more (e.g., 99%) of the particles are of an average particle size of 1 μm or less.

### Exemplary Bioactive Glasses are Described Below

### Bioglass 4555

Bioglass form on 45S5, or bioglass 45S5, is a bioactive glass that is composed of 49.5% Si02, 17.0% NaO, 26.9% Cao, and 6.6% P20s, in weight %. In certain embodiments, bioglass 45S55 is a powder, and can be provided as a dry powder. In these embodiments, the bioglass 45S5 powder is composed of nanoparticles with an average particle size of 1 μm or less, preferably 0.5 μm. An average particle size of 1 μm or less allows the bioactive glass powder to penetrate into the lumens of dentinal tubules that have diameters of approximately 0.8 μm to 1 μm before etching and may have diameters of approximately 1 μm or more after etching. In addition, an average particle size of 1 μm or less can facilitate penetration of the bioactive glass into the partially etched intertubular dentin that has openings of less than 1 μm. Bioglass 45S5 powder may be prepared by methods known to those of skill in the art, including, but not limited to, planetary ball milling of bioglass 45S5 glass chips.

### F Glass

BioGlass F or F Glass is a bioactive glass that has the following approximate composition by weight percentage: Si02 (44%), Na20 (23%) CaO (10%), MgO (4.5%), P205 (6%), and CaF2 (12.5%). In certain embodiments, F glass is a powder, and can be provided as a dry powder. In these embodiments, the F glass powder may be composed of nanoparticles with an average particle size of 1 μm or less, preferably 0.5 μm. An average particle size of 1 μm of less allows the bioactive glass powder to penetrate the lumens of dentinal tubules, that have diameters of approximately 0.8 to 1 μm before etching and may have diameters of approximately 1 μm or more after etching. In addition, an average particle size of 1 μm or less may allow the bioactive glass to penetrate the partially etched intertubular dentin that has openings of less than 1 μm. F glass powder may be prepared by methods known to those of skill in the art, including, but not limited to, planetary ball milling of chips of F glass.

Bioglass 45S5 Bioglass F powders may be prepared by methods known to those of skill in the art, including but not limited to planetary ball milling of Bioglass 45S5 glass chips.

### Solvents

The solvent used to disperse the bioactive glass in the dental whitening composition can be any suitable solvent available in the art. As described above, bioactive glasses elicit a series of chemical reactions when they are brought into contact with tissue or any aqueous environment that contains calcium and phosphate, leading to the formation of carbonated hydroxyapatite (HCA). Accordingly the solvent can be described as a “non-aqueous solvent” referring to solvents that do not contain water as a predominant component, and include, for example, solvents that contain less than 10% water by volume, less than 5% water by volume, less than 1% water by volume, and may contain no detectable water. Such non-aqueous solvents, thus, have a water content that is sufficiently low to avoid reaction of the bioactive glass so as to significantly generate silanol groups. Thus in some cases, the solvent for the adhesive composition contains less than 5% to less than 1% water, and an substantially lack water. In certain embodiments, the solvent is an alcohol. In these embodiments, the alcohol solvent may be ethanol, isopropyl alcohol, or any other suitable alcohol in other cases, suitable solvents may include acetone. In some cases, the non-aqueous solvent is other than acetone.

The amount of solvent used in the dental whitening composition can vary according to the desired properties of the composition. For example, solvent can be added or removed (e.g., by vacuum or evaporation), so as to provide a final dental whitening composition having a desired viscosity or consistency. For example, the composition can be flowable at ambient temperature, and may be of a consistency compatible with painting the composition onto the surface to be treated. Exemplary compositions can have the consistency of a fluid paste or gel. In general, the viscosity of the composition is compatible with its so as to allow the composition to penetrate to a sufficient degree into the dentin matrix.

### Bioactive Glass-Containing Dental Whitening Compositions or Use in Preparation and Whitening Methods

As noted above, dental whitening compositions containing bioactive glass can include compounds to facilitate one or more steps of a dental whitening method according to the present disclosure. Thus, dental whitening compositions can provide for activity as one or more of an etchant (as in, etching of dentin), a suitable non-aqueous solvent, bioglass (to facilitate whitening of a composite to dentin), or combinations thereof (e.g., to provide for any combination (including all) steps of dental whitening methods). In these embodiments, the dental etchant may be an inorganic or organic acid, such as but riot limited to phosphoric acid, maleic acid, or citric acid.

For example, in one embodiment, the dental whitening composition carp include a primer, a bioactive glass, and a non-aqueous solvent. In these embodiments, the dental whitening composition find use in methods that include the sequential steps of etching; and contacting the etched surface of dentin with the dental whitening composition, such that the dental whitening composition is applied to the etched surface of dentin. Thus, for example, the dental whitening composition includes a bioactive glass and a non-aqueous solvent. In these embodiments, the dental whitening composition may find use in methods that include contacting a tooth with the dental whitening composition, such that the steps of etching and priming are achieved in a single application. In any of these embodiments, the method may further include contacting the etched and primed surface of dentin with an adhesive to facilitate the application of the dental whitening composition as part of the restoration of teeth. Additionally, the dental whitening composition will whiten the dentin and when the teeth are properly restored using a dental resin composite the underling whiter dentin will make the restored tooth appear whiter.

In other embodiments, the dental whitening composition includes a primer, a bioactive glass and a non-aqueous solvent. In these embodiments, the dental whitening composition may find use in methods that include contacting a tooth with an etchant to provide an etched dentin surface, and contacting the etched dentin with the dental whitening composition, such that the steps of priming and whitening are achieved in a single application step.

In other embodiments, the dental whitening composition includes a suspension (e.g., slurry) of a bioactive glass in a non-aqueous solvent. In these embodiments, the dental whitening composition may find use in methods that include the steps of etching; contacting the etched surface of dentin with the dental whitening composition; priming; and whitening.

In any of the above embodiments, the solvent may be an alcohol, such as, but not limited to, ethanol.

### Methods of Making

Methods for making a dental whitening composition for whitening, in general, can involve combining a bioactive as described above with a suitable non-aqueous solvent (to generate a slurry).

In certain cases, the method includes mixing a bioactive glass, as described above, such that it is combined with a non-aqueous solvent (e.g., acetone or an alcohol, such as, but not limited to, ethanol) prior to combining with the bioactive glass.

The types and amounts of bioactive glass, as well as the types of solvents, can be those as exemplified herein.

The methods for production of a dental whitening composition contemplate production of intermediate compositions, that can later be combined to form the final dental whitening composition. For example, the bioactive glass can be provided in a non-aqueous solvent in a first container. In another example, the bioactive glass can be provided as a powder in first container and a non-aqueous solvent provided in second container. All or part of the contents of the two containers can then be combined prior to use, according to directions which can be optionally packaged with the containers.

### Methods of Use

The dental whitening compositions described herein may be used in dental restoration procedures to restore the whiteness of the tooth or teeth when there has been a significant loss of enamel and the dentin is exposed. Generally, the steps involved for whitening a tooth include etching the tooth, applying the priming and bioglass mixture to the tooth, so that it is infused into the dentinal tubules.

Effectively whitening of the tooth may require preparation of the tooth prior to whitening due to the hydrophilic nature of dental tissue, such as dentin. To facilitate the formation of a mechanical bond between the bioglass and dentin, the steps described herein of etching, priming, and whitening may be used.

Etching involves applying an acid to the surface of a tooth to superficially demineralize the spa tits of dentin. Etching may also remove surface contaminant also known as the“smear layer,” on the surface of dentin. Etching dentin exposes a layer of collagen fibers. Removal of the smear layer also exposes the dentinal tubules. Etching creases the surface area available for whitening and facilitates penetration of the bioglass into the porosities in the dentin revealed by the etching procedure. Penetration of the bioglass into the dentin matrix a hybrid layer that is composed of the dentin and the bioglass. This facilitates the formation of a mechanical bond between the dentin tubulars and the bioglass. Dental etchants may be an inorganic or organic acid such as, but not limited to, phosphoric acid, maleic acid, citric acid.

The subsequent step of whitening the dental resin composite to the dentin with the dental whitening composition may include contacting the etched surface of dentin with the dental whitening composition. Prior to contacting the etched and/or primed surface of dentin with the dental whitening composition, the dental whitening composition may be made as described herein. In some cases, the dental whitening composition may be made in advance and stored until used. The dental whitening composition may be stored in a sealed container, such that, during storage, the dental whitening composition remains substantially free of water.

### Application of Bioactive Glass-Containing Suspension

In certain embodiments, the method includes the steps of etching, contacting the etched surface of dentin a suspension of a bioactive glass in a non-aqueous solvent, and whitening. In these embodiments, the dentin is first etched with a dental etchant, as described herein. In these embodiments, the dental etchant may be an inorganic or organic acid such as, but not limited to, phosphoric acid, maleic acid, or citric acid. Then, the etched dentin is contacted with a suspension of a bioactive glass (e.g., slurry) in a non-aqueous solvent. In some cases, the non-aqueous solvent may be an alcohol, such as, but not limited to, ethanol. Subsequently, the tooth with bioactive glass may be contacted with a dental primer and cured. Then, the primed surface of the dentin may be contacted with an adhesive. The adhesive may be cured as described herein. In some cases, the method also includes contacting the adhesive layer with a dental resin composite such that the dental resin composite is adhered to the dentin.

### Kits

Also of interest are kits for use in practicing certain embodiments of the invention. The components of the kits can be adapted for use in any of the various methods described herein.

In certain embodiments, the kits can include a dental whitening composition of the present disclosure that may be provided for immediate use, or may be provided as separate components to be combined to form the dental whitening composition. The dental whitening composition can be provided in a container that can be adapted to facilitate application to an etched dentin surface. For example, the container can be in the form of a needleless syringe fitted with a plunger to provide for application of the contents of the container to an etched dentin surface.

For example, where the dental whitening composition is provided as separate components (e.g., as described above in the context of methods of making the dental whitening composition), the kit can include a first container containing a bioactive glass (e.g., powder) and a second container with a non-aqueous solvent.

Optionally, the kit can include instructions for combining all or a portion of the first and second containers to provide a dental whitening composition. Provided in a second container, all or part of the contents of the two containers can then be combined prior to use, according to directions that can be optionally provided with the kit. In one embodiment, where the dental whitening composition is provided as separate components to be combined prior to use, the two components are maintained in separate containers that are not in fluid communication, but that are separated by a frangible or removable wall, that can be broken, or removed, to facilitate mixing of the two components prior to use.

The kits can optionally include additional components. For example, the kits can include one or both of an etchant and a primer compatible for use with the dental whitening composition. Such additional components can be provided in additional containers as may be desired.

In certain embodiments, the kit may include an etchant and a dental whitening composition of bioglass, where the etchant and the dental whitening composition are provided in separate containers. In these embodiments, the dental whitening composition may include a bioactive glass and a solvent.

In other embodiments the kit may include a dental whitening composition provided in a single container. In some cases, the dental whitening composition may include a bioactive glass and a solvent.

In certain embodiments, the kits will further include instructions for practicing the subject methods or means for obtaining the same (e.g., a website URL directing the user to a webpage that provides the instructions), where these instructions may be printed on a substrate, where substrate may be one or more of a package insert, the packaging, reagent containers, and the like. In the subject kits, the one or more components are present in the same or different containers, as may be convenient or desirable.

### EXAMPLES

The following examples are put forth, so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Celsius, and pressure is at or near atmospheric.

### Example 1

Studies to test the whit of dentin in the presence of bioglass 45S5 were performed.

### Preparation of Tooth Samples

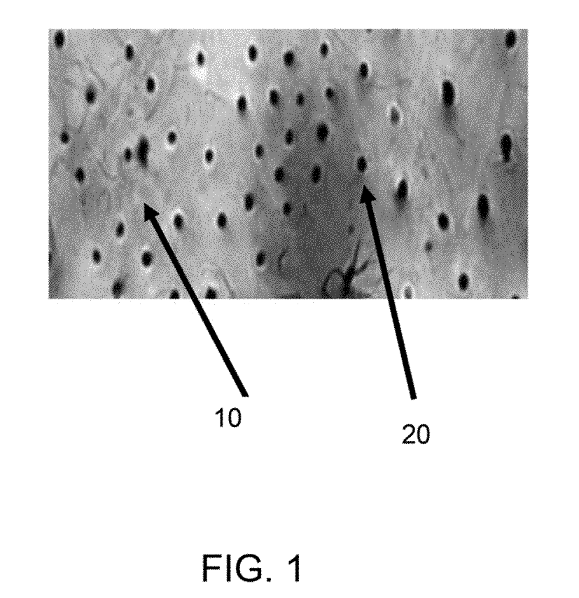

The occlusal enamel of six human third molars were removed using a belt sander with 240-grit silicon carbide paper, following which the roots were removed using a slow-speed saw (IsoMet®, Buehler Ltd., Lake Bluff, Ill.) with water coolant to produce tooth discs approx 5 mm thick. The exposed dentin was polished with 320-grit silicon carbide paper. The discs were mounted on open-ended tubes with hot glue and attached to a vacuum trap. The color of each of the samples was compared to the uncoated Panatone® color guide. The color comparison results are shown in Table 1. Scanning electron micrographs were taken of the Sample 1 (shown in FIG. 1) presenting the dentin 10 and the dentinal tubule 20.

TABLE 1

| | | |
|---|---|---|
| Sample No. | Initial color | Final color |

| | |
|---|---|
| 1 | OS 2-1 U |
| 2 | OS 2-2 U |
| 3 | OS 2-2U |
| 4 | OS 2-1 U |
| 5 | OS 2-4 U |
| 6 | OS2-2 U |

The occlusal dentin was etched for 15 seconds with Scotchbond™ gel etchant (3M/ESPE, St. Paul, Minn.) and rinsed for a further 15 seconds with deionized water. A slurry of 20% or 40% (w/v) bioactive glass of the formulation 45S5 (Bioglass 45S5, SEM-COM, Toledo, Ohio) in ethanol was applied to the top of the sample. The average particle size of the ground bioactive glass powder was less than 1 μm, and it was prepared by planetary ball milling of glass chips. The glass slurry was prepared with ethanol. Vacuum was applied at 530 mm Hg for one minute; the sample surface was kept moist by re-applying the slurry every few seconds. After removal from the vacuum, any excess solid was gently rinsed away with deionized water.

### Results

Following application of the bioglass, the color of each of the samples was compared to the uncoated Panetone color guide. Color comparison results are shown in Table 2.

TABLE 2

| Sample No. | Initial color | Final color |
|---|---|---|
| 1 | OS 2-1 U | OS 2-7 U |
| 2 | OS 2-2 U | OS 2-6 U |
| 3 | OS 2-2U | OS 2-8 U |
| 4 | OS 2-1 U | OS 2-9 U |
| 5 | OS 2-4 U | OS 2-6 U |
| 6 | OS2-2 U | DS 2-8 U |

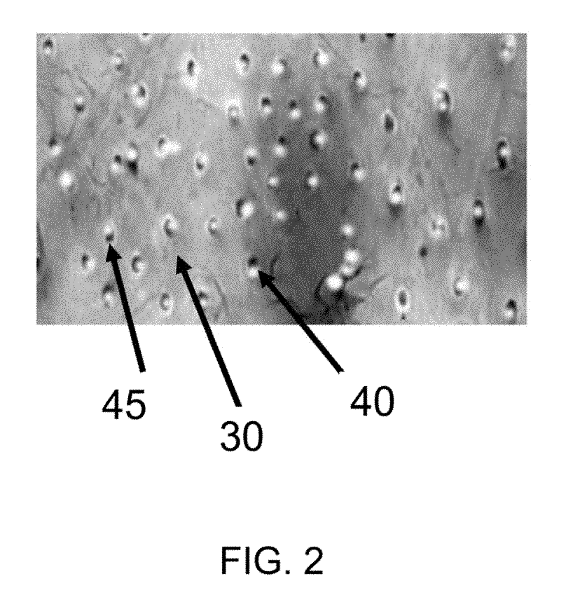

Second scanning electron micrographs of Sample 1 taken are shown in FIG. 2 (dentin 30 and dentinal tubule 40) with a white bioglass particle 45 in the tubule.

The above experiments showed that the bioactive glass are effectively incorporated into the resin-dentin whitening process. Once incorporated into the dentin, the microparticles of bioactive glass lighten the samples.

### Example 2

Studies to Test the Whitening of Dentin in the Presence of F Glass were Performed.

### Preparation of Tooth Samples

The occlusal enamel of six human third molars were removed using a belt sander with 240-grit silicon carbide paper, following which the roots were removed using a slow-speed saw (IsoMet, Buehler Ltd., Lake Bluff, Ill.) with water coolant to produce tooth discs approximately 5 mm thick. The exposed dentin was polished with 320-grit silicon carbide paper. The discs were mounted on open-ended tubes with hot glue, and then attached to a vacuum trap. The color of each of the samples was compared to the uncoated Pantone color guide. Color comparison results are shown in Table 3.

TABLE 3

| | | |
|---|---|---|
| Sample No. | Initial color | Final color |

| | |
|---|---|
| 7 | OS 2-2 U |
| 8 | OS 2-2 U |
| 9 | OS 2-1 U |
| 10 | OS 2-2 U |
| 11 | OS 2-4 U |
| 12 | OS2-2 U |

The occlusal dentin was etched for 15 seconds with Scotchbond gel etchant (3M/ESPE, St. Paul, Minn.) and rinsed for a further 15 seconds with deionized water. A slurry of 20% or 40% (w/v) bioactive glass of the formulation Bioglass F or F Glass and has the following approximate composition by weight percentage: SiO2 (44%), Na2O (23%), Cao (10%), MgO (4.5%), P2Os (6%), CaF2 (12.5%) in ethanol was applied to the top of the sample. The average particle size of the ground bioactive glass powder was approximately 0.5 μm, and it was prepared by planetary ball milling of glass chips. The glass slurry was prepared with ethanol. Vacuum was applied at 530 mm Hg for one minute; the sample surface was kept moist by re-applying the slurry every few seconds. After removal from the vacuum, any excess solid was gently rinsed away with deionized water.

### Results

After the application of the bioglass the color of each of the samples was compared to the uncoated Pantone color guide. The color comparison results are shown in Table 4

TABLE 4

| Sample No. | Initial color | First application color |
|---|---|---|
| 7 | OS 2-2 U | OS 1-8 U |
| 8 | OS 2-2 U | OS 2-8 U |
| 9 | OS 2-1 U | OS 2-9 U |
| 10 | OS 2-2 U | OS 2-7 U |
| 11 | OS 2-4 U | OS 2-8 U |
| 12 | OS2-2 U | OS 2-9 U |

The application of Bioglass F mixture was repeated. The glass slurry previously prepared with ethanol was applied to the surface. Vacuum was applied at 530 mm Hg for one minute; the sample surface was kept moist by re-applying the slurry every few seconds. After removal from the vacuum any excess solid was gently rinsed away with deionized water. The color comparison results are shown in Table 5

TABLE 5

| Sample No. | Initial color | Second application color |
|---|---|---|
| 7 | OS 2-2 U | OS 1-9 U |
| 8 | OS 2-2 U | OS 1-8 U |
| 9 | OS 2-1 U | OS 1-9 U |
| 10 | OS 2-2 U | OS 1-8 U |
| 11 | OS 2-4 U | OS 1-8 U |
| 12 | OS2-2 U | OS 1-9 U |

The above experiments showed that the bioactive glass are effectively incorporated into the resin-dentin whitening process. Once incorporated into the dentin, the microparticles of bioactive glass lighten the samples and that multiple applications enhanced the lightening the color.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

We claim:

1. A method whitening a tooth having exposed dentin comprising of the steps: applying an etching composition comprising an etchant to said tooth exposed dentin to produce an etched dentin surface,
letting said tooth dry,
applying a composition that comprises of a bioactive glass substantially lacking silanol groups and a non-aqueous solvent to said tooth etched dentin surface to form a first layer of bioactive glass on said etched dentin surface,
letting said tooth dry.

2. The method wherein the tooth of claim 1 is whitened comprising of the steps: applying a composition that comprises of a bioactive glass substantially lacking
silanol groups and a non-aqueous solvent to said tooth on top of said first layer to form a second layer of bioactive glass on said etched dentin surface,
letting said tooth dry.

3. The method of claim 1, wherein said etching composition and said bioactive glass composition are optionally not applied in separate steps.

4. The method of claim 1, wherein said bioactive glass is present at about 0.5% to 10% by weight percentage of the non-aqueous solvent composition.

5. The method of claim 1, wherein said bioactive glass has the following approximate composition by weight percentage: SiO2 (44%), Na2O (23%), Cao (10%), MgO (4.5%), P2Os (6%), CaF2 (12.5).

6. The method of claim 1, wherein said bioactive glass has the following approximate composition by weight percentage: SiO2 (49.5%), Na2O (17%), CaO (26.9%), P2Os (6.6%).

7. The method of claim 1, wherein said bioactive glass has an average particle size of 1 μm or less.

8. The method of claim 1, wherein said bioactive glass has an average particle size of 0.5 μm or less.

9. The method of claim 1, wherein said non-aqueous solvent is an alcohol.

10. The method of claim 9, wherein said alcohol is ethanol.

11. A method whitening a tooth having exposed dentin surface comprising of the steps: applying mixture of an etching composition comprising an etchant to the said exposed dentin on said tooth to produce an etched dentin surface; applying a bioactive glass composition comprising a bioactive glass substantially lacking silanol groups and a non-aqueous solvent to said etched dentin surface to whiten said surface and then applying a dental resin composite to said tooth and said etched dentin surface.

12. The method of claim 11, wherein said bioactive glass is present at about 0.5% to 40% by weight percentage of the bioactive glass composition.

13. The method of claim 11, wherein said bioactive glass has the following approximate composition by weight percentage: SiO2 (44%), Na2O (23%), Cao (10%), MgO (4.5%), P2Os (6%), CaF2 (12.5%).

14. The method of claim 11, wherein said bioactive glass has the following approximate composition by weight percentage: SiO2 (49.5%), Na2O (17%), Cao (26.9%), P2Os (6.6%).

15. The method of claim 11, wherein said bioactive glass has an average particle size of 1 μm or less.

16. The method of wherein said bioactive glass hasan average particle size of 0.5 μm or less.

17. The method of claim 11, wherein said non-aqueous solvent is an alcohol.

18. The method of claim 17, wherein said alcohol is ethanol.

19. A kit comprising: a dental whitening composition comprising a bioactive glass substantially lacking silanol groups; a non-aqueous solvent comprising an alcohol; optionally, an etching composition suitable for use with the dental whitening composition.

20. The kit according to claim 19, wherein said dental whitening composition is provided as separate components, wherein a first component comprises the bioactive glass in the non-aqueous solvent, wherein the said first component is provided in a first container; and a second component comprises of an etching composition, wherein the said second component is provided in a second container.
